(19) United States
(12) Patent Application Publication
(10) Pub. No.: US 2020/0174448 A1
(43) Pub. Date: Jun. 4, 2020

(54) Method of Providing Custom Limb Rehabilitation Devices for Animals by Executing Computer-Executable Instructions Stored on a Non-Transitory Computer-Readable Medium

(71) Applicant: Rosemary Christine Koop, Oregon City, OR (US)

(72) Inventor: Rosemary Christine Koop, Oregon City, OR (US)

(21) Appl. No.: 16/607,555

(22) PCT Filed: Oct. 5, 2017
(86) PCT No.: PCT/IB2017/056164

§ 371 Date: Oct. 23, 2019

Related U.S. Application Data
(60) Provisional application No. 62/404,453, filed on Oct. 5, 2016.

(51) Int. Cl. G05B 19/4099; A61D 9/00; B33Y 50/02; B29C 64/393; B33Y 80/00; B29L 31/00; A61F 13/04; A61F 5/01

(52) U.S. Cl. CPC G05B 19/4099; A61D 9/00; B33Y 50/02; B29C 64/393; A61F 5/0111; B29L 2031/753; G05B 2219/35134; A61F 13/04; G05B 2219/49023; B33Y 80/00

(57) ABSTRACT

A method of providing custom limb rehabilitation devices for animals provides a software application for an end user to submit subject attributes and injury data for an injured animal in order to procure a custom brace or cast for the animal. The subject attributes and injury data are processed in order to produce a digital limb rehabilitation model, which is then used to create a physical counterpart of the digital model through an additive manufacturing.

The current application is a 371 of international Patent Cooperation Treaty (PCT) application PCT/IB2017/056164 filed on Oct. 5, 2017. The PCT application PCT/IB2017/056164 claims a priority to a U.S. provisional application Ser. No. 62/404,453 filed on Oct. 5, 2016.

### FIELD OF THE INVENTION

The present invention relates generally to orthotics. More particularly, the present invention relates to producing custom casts or braces for treatment of animal injuries.

### BACKGROUND OF THE INVENTION

Orthotics is a specialty within the medical field concerned with the design, manufacture, and application of orthoses, which are externally applied devices used to modify the structural and functional characteristics of the neuromuscular and skeletal system. An orthosis may be used to control, guide, limit and/or immobilize an extremity, joint or body segment, restrict movement in a given direction, assist general movement, or aid rehabilitation from fractures after removal of a cast, among other functions.

Animals may benefit from the use of an orthosis upon suffering an injury to a lower limb or paw, such as a fracture, torn meniscus, ruptured Achilles tended, or injured cruciate ligament. An animal may also have an orthopedic condition due to arthritis, spinal cord injury, or a congenital abnormality.

If an animal is injured, the animal may require evaluation by a veterinarian to determine if they will require a cast or brace, depending on the severity of the injury. If the injury does necessitate a cast or brace, the animal has to be measured in order to receive an accurately-sized brace or cast. This process takes a considerable amount of time which further lengthens the overall recovery time for the animal.

It is therefore an objective of the present invention to provide a system and method for providing custom animal braces and casts. The present invention includes a software application where a user may provide information on the animal such as the breed and size of the animal and also upload files such as pictures of the animal's injury. The system will take all the information and files provided by the user into account and diagnose if the animal will require a cast or brace for the injury or the user will be able to input a veterinaries diagnosis. If the animal does require a brace or cast, the system will then send inputs to a 3D printer to print out a custom brace or cast for the animal. The custom brace or cast will include fasteners to secure onto a body part of animal and may be also be accessorized. The system will do all this in quick fashion reducing the overall recovery time for the animal and also providing an accurately-sized brace or cast for the animal.

### BRIEF DESCRIPTION OF THE DRAWINGS

FIG. 1 is a process flow diagram of the present invention.

FIG. 2 is a continuation of FIG. 1.

FIG. 3 is a stepwise flow diagram of the general method of the present invention.

FIG. 4 is a stepwise flow diagram showing steps for defining the limb rehabilitation device as a limb brace or a limb cast.

FIG. 5 is a stepwise flow diagram showing steps for receiving and processing injury data.

FIG. 6 is a stepwise flow diagram showing steps for executing the additive manufacturing process.

FIG. 7 is a stepwise flow diagram showing steps for choosing surgical applications.

FIG. 8 is a perspective view of an example of a custom brace produced through the present invention.

FIG. 9 is a front view of an example of a custom brace produced through the present invention.

FIG. 10 is a side view of an example of a custom brace produced through the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

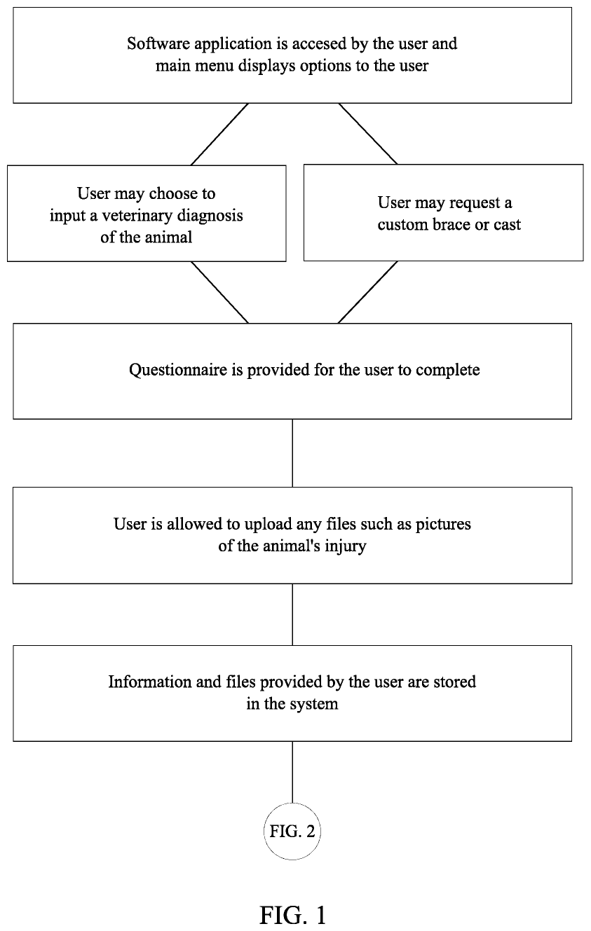

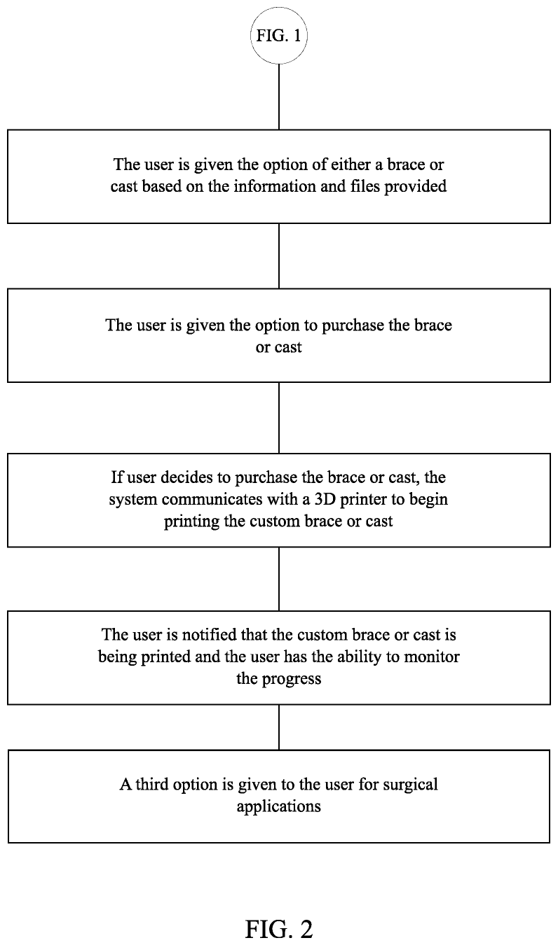

The present invention is a system and method for more easily providing custom animal braces and casts for injury care and rehabilitation based on information provided by the caretaker of the animal or other responsible party. The present invention is directed primarily for use pertaining to cats and dogs, though it is contemplated that the present invention may be utilized in various applications where the functionality of the present invention may be applicable to other animals, or even humans. The present invention is preferably enacted as a software application or digital platform that enables a user to submit relevant information about the subject animal in order to commission a brace, cast or similar device for stabilization and rehabilitation of an injured limb of an animal. The present invention is preferably embodied as a software application which can be accessed on a personal computer and/or a mobile device such as a smartphone, tablet, or similar device. In the preferred embodiment of the present invention, the system is a mobile application which may be accessed on any mobile device. The system will provide a menu for the user where a user can request a diagnosis for a physical injury of an animal or input a diagnosis of a veterinary and request a custom brace or cast. A general overview of the process of the present invention is shown in FIGS. 1-2.

In general, a method of providing custom limb rehabilitation devices for animals is disclosed. It may be understood to the reader that the focus of the present invention is upon steps taken in a method executed by one or more various electronic processing devices by executing computer-executable instructions stored on a non-transitory computer readable medium, and may be further supplemented by actions performed by human actors after the primary disclosure of the present invention is accomplished. The present invention may further or alternatively be understood as a system of devices that are coordinated through electronic communication and instruction and user input in order to accomplish the purpose of the present invention.

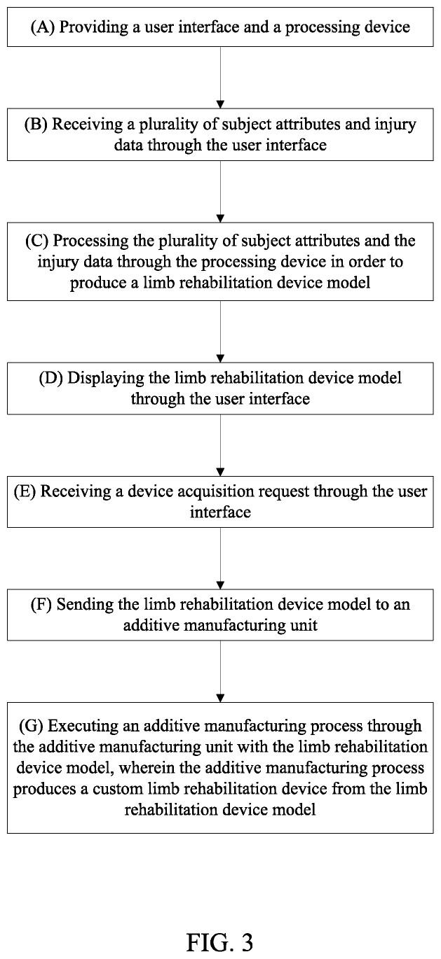

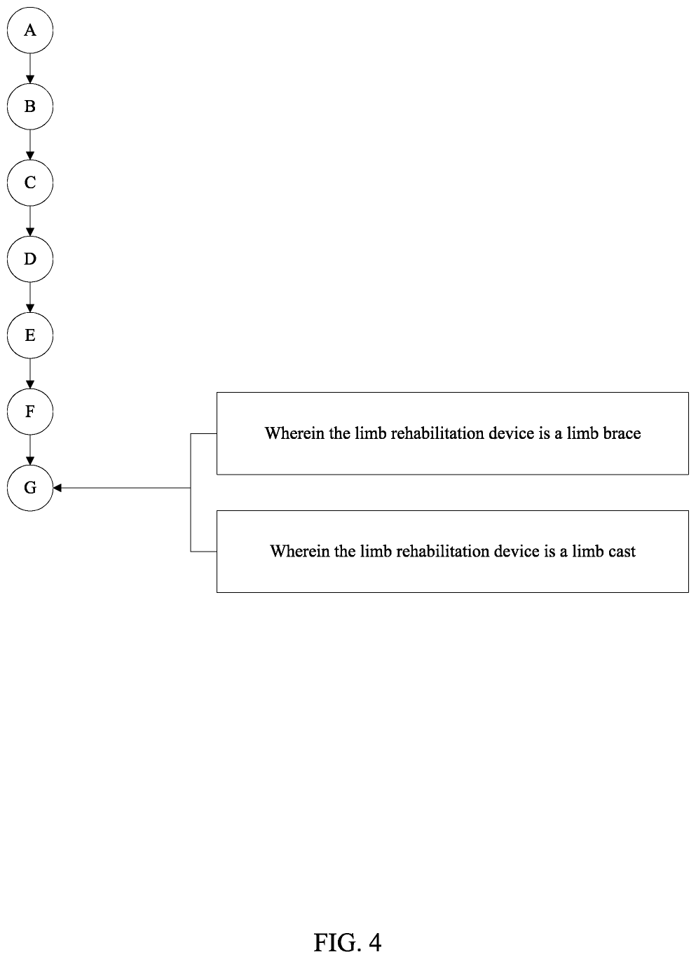

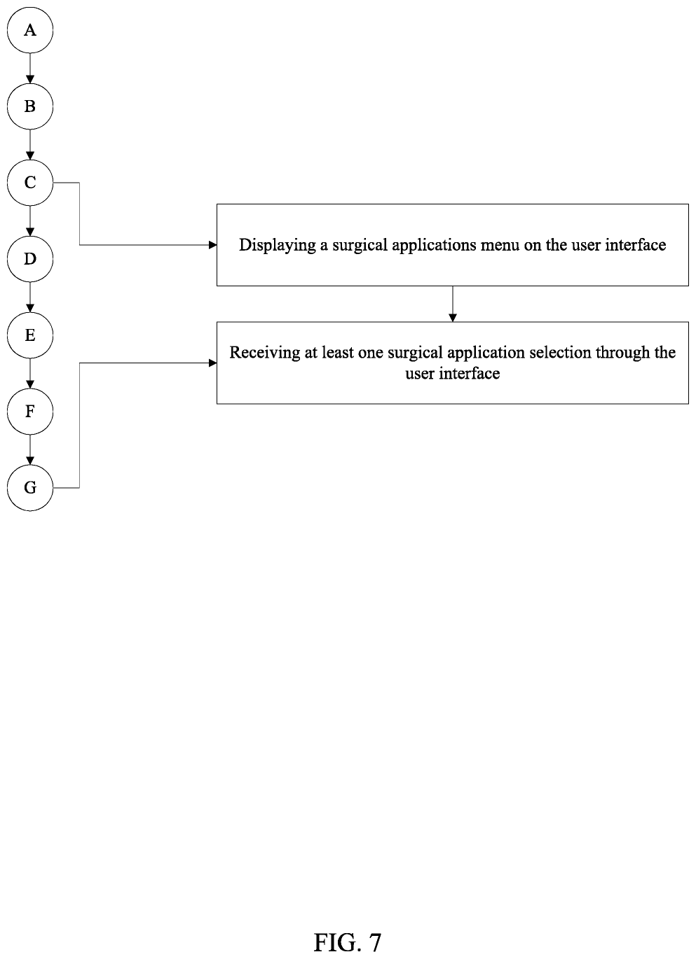

Referring to FIGS. 3-7, in the general method of the present invention, a user interface is provided, as is a processing device. The user interface may be understood to be an electronic device or a means of interacting with an electronic device to the purpose of submitting relevant information, requests, and instructions from the caretaker of the injured animal in question to the processing device, which may be understood to be, but is not limited to, a central processing hub, server, cloud system or other general means of processing received electronic input in order to produce appropriate electronic output as relevant or necessary to accomplish the spirit of the present invention.

In some embodiments, the present invention may be presented to the end user as a digital application on a smartphone, tablet, laptop, personal computer, or similar electronic device. As such, the present invention will present through the user device a user interface through which the end user will interact with in order to utilize the present invention. In some embodiments, the user interface may be understood to be a collection of digital programming code compiled into a visual representation and presented to the user on a digital display screen, enabling the user to interact with various functions of the end user application of the present invention. In some embodiments, the user interface may be understood to be one or more physical input devices through which the user is able to communicate relevant information and instructions to the processing device of the present invention.

To begin the process, the user initializes the application of the present invention. For example, the user may launch a smartphone application embodiment of the present invention on their smartphone or other mobile device. The user may be presented with a menu comprising multiple options for proceeding. In general, however, the user will input a plurality of subject attributes as well as injury data through the user interface.

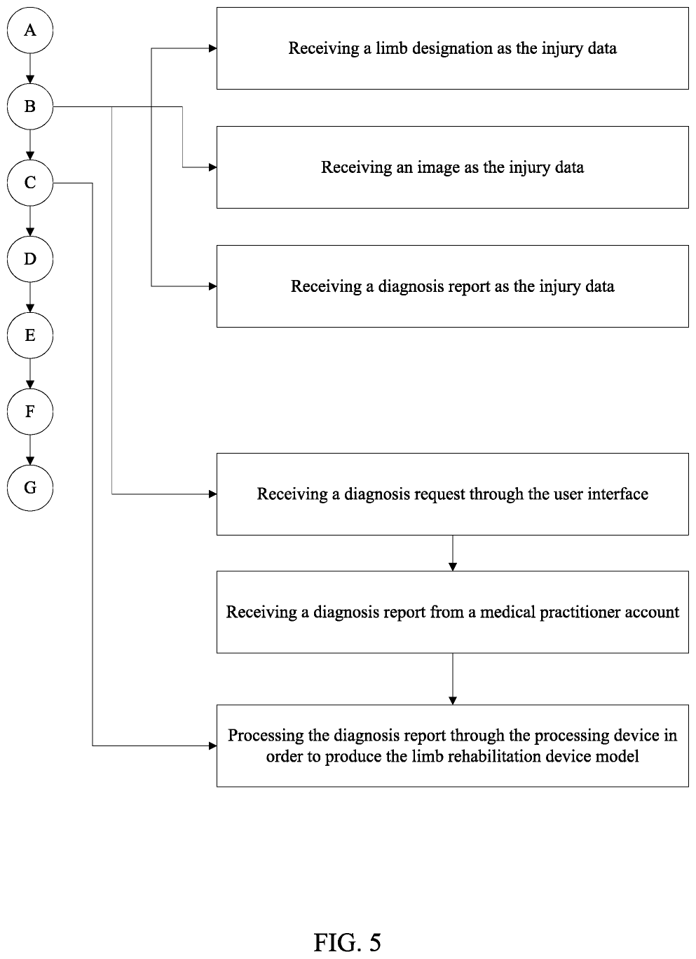

As illustrated in FIG. 5, the injury data may take various forms, such as, but not limited to, a limb designation, one or more images, or a diagnosis report. In some embodiments, the end user may submit a diagnosis report previously acquired from a medical professional such as a veterinarian.

Furthermore, in some embodiments, the user may request a diagnosis report through the present invention after submitting injury data such as a limb designation, a written description, one or more images of the injury to the animal, and the like. In such a case, a diagnosis request is received through the user interface and sent to the processing device. The processing device may then notify an appropriate user, such as a veterinarian, of the diagnosis request. The veterinarian or other relevant personnel will then review the submitted injury data for the animal, produce a diagnosis report, and submit the diagnosis report to the processing device of the present invention. Thus, a diagnosis report may be received from a medical practitioner account. Subsequently, the diagnosis report is processed through the processing device as previously disclosed in order to produce the limb rehabilitation device model.

The plurality of subject attributes and the injury data are received through the interface, sent to the processing device, and processed through the processing device in order to produce a limb rehabilitation device model. In some embodiments, the limb rehabilitation device may be a limb brace. In some embodiments, the limb rehabilitation device may be a limb cast. In some embodiments, the user may be allowed to select from either a limb brace, a limb cast, or another similar type of device as the limb rehabilitation device. The limb rehabilitation device model may be understood to be a digital construction of a potential brace or cast that is created based on the submitted subject attributes of the subject animal, such as, but not limited to, type, breed, weight, size of animal, limb dimensions, as well as any other standard or non-standard questions applicable to the present invention. The user may be presented with a questionnaire through the user interface into which the user enters the aforementioned parameters.

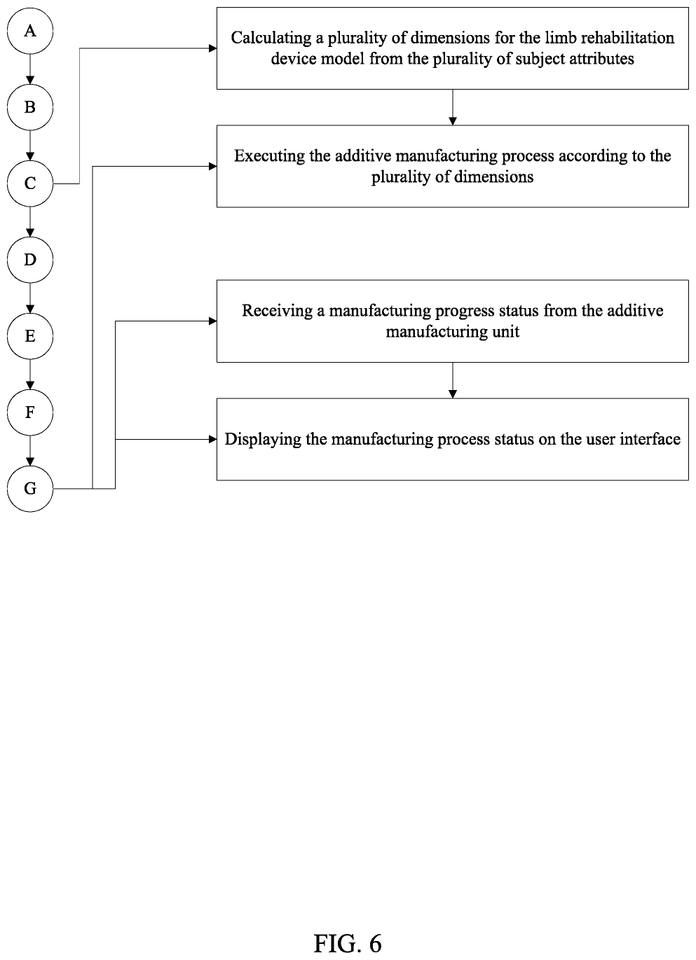

It is contemplated that a variety of means of processing the plurality of subject attributes and injury data may be implemented in order to accomplish the purpose of the present invention. Specific algorithms or other means for said purpose are not herein disclosed, and may be understood to be any currently existing or new methods, processes, algorithms, or other relevant means to produce the limb rehabilitation device model. The focus of the present invention is on the general process of receiving relevant subject attributes and injury data, using said attributes and injury data to generate a digital model for a limb rehabilitation device, and subsequently creating a physical version of the digital model utilizing a three-dimensional (3D) printer or other additive manufacturing technology. In any case, a plurality of dimensions are calculated for the limb rehabilitation device model from the plurality of subject attributes, and/or one or more components of the injury data, and/or other relevant sources of information. The additive manufacturing process is then executed according to the plurality of dimensions, as shown in FIG. 6.

After the plurality of subject attributes and the injury data are processed through the processing device in order to produce the limb rehabilitation model, the limb rehabilitation device model may be displayed to the user through the user interface for approval.

The user may then choose to place an order for a physical version of the limb rehabilitation device model. If so, a device acquisition request is received through the user interface in order to request a physical version of the limb rehabilitation device model. The user will then be requested to provide their shipping and billing information for the purchase. The limb rehabilitation device model is then sent to an additive manufacturing unit such as, but not limited to, a 3D printer. Then, an additive manufacturing process is executed through the additive manufacturing unit with the limb rehabilitation device model, such that the additive manufacturing process produces a custom limb rehabilitation device from the limb rehabilitation device model. In some embodiments, the user may be provided with the option to monitor the progress of the additive manufacturing process. Thus, a manufacturing progress status may be received from the additive manufacturing unit, and the manufacturing progress status may be displayed to the user on the user interface.

Furthermore, in some embodiments, the user may be presented with a menu for selecting various surgical applications. Thus, a surgical applications menu may be displayed to the user on the user interface, and at least one surgical application selection may be received through the user interface from the user. The surgical applications may vary in nature, and may include items such as ordering one or more specific treatments or procedures to treat the injury to the animal, as well as ordering prescriptions. The surgical applications menu will provide a description of each surgical application and provide the user the option to choose one or multiple surgical applications.

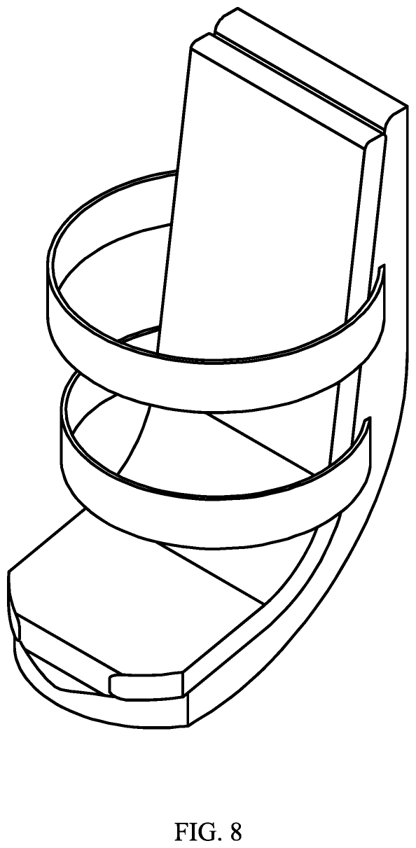

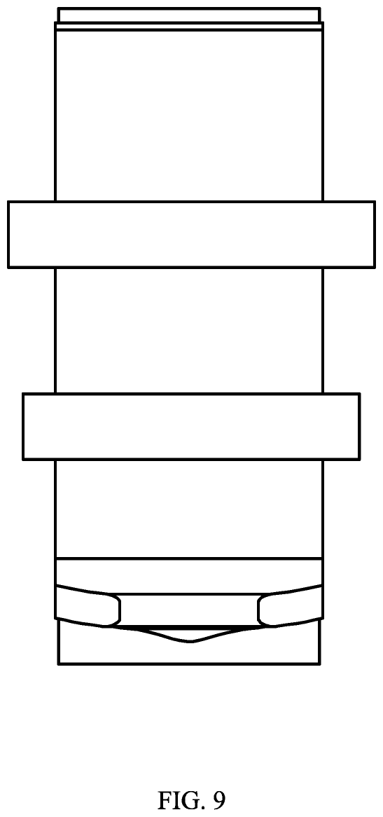

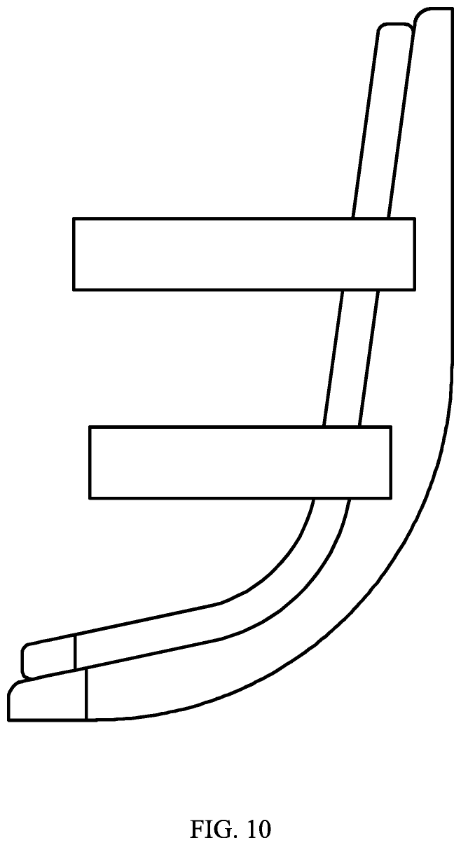

In some embodiments, the limb rehabilitation device comprises a 3D printed piece, a padding, and a plurality of fasteners. FIGS. 8-10 show example illustrations of the one embodiment of the final product. The 3D printed piece may be any size and shape. The 3D printed piece may be composed of any material. The 3D printed piece is printed using a 3D printer in communication with the system of the present invention. The 3D printed piece is shaped and sized according to the information provided by the user to the system. The padding is adhered to front of the 3D printed piece. In an alternative embodiment of the present invention, the padding comprises a plurality of layers. The plurality of layers will provide more cushion to the padding. The plurality of fasteners may be any type of fastener able to secure the custom brace or case to a body part of an animal. In one embodiment of the present invention, the plurality of fasteners is adjustable straps. The plurality of fasteners is positioned at the sides of the 3D printed piece and extend towards the front. In another embodiment of the present invention, the plurality of fasteners may be clamps. Other accessories may be added to the custom brace or cast. The padding, fasteners, layers, straps, and any other accessories may need to be added to the 3D printed piece manually by a personal affiliated with the operation of the present invention.

Although the invention has been explained in relation to its preferred embodiment, it is to be understood that many other possible modifications and variations can be made without departing from the spirit and scope of the invention as hereinafter claimed.

What is claimed is:

1. A method of providing custom limb rehabilitation devices for animals by executing computer-executable instructions stored on a non-transitory computer-readable medium comprises the steps of:
providing a user interface and a processing device;
receiving a plurality of subject attributes and injury data through the user interface;
processing the plurality of subject attributes and the injury data through the processing device in order to produce a limb rehabilitation device model;
displaying the limb rehabilitation device model through the user interface;
receiving a device acquisition request through the user interface;
sending the limb rehabilitation device model to an additive manufacturing unit; and
executing an additive manufacturing process through the additive manufacturing unit with the limb rehabilitation device model, wherein the additive manufacturing process produces a custom limb rehabilitation device from the limb rehabilitation device model.

2. The method of providing custom limb rehabilitation devices for animals by executing computer-executable instructions stored on a non-transitory computer-readable medium as claimed in claim 1, wherein the limb rehabilitation device is a limb brace.

3. The method of providing custom limb rehabilitation devices for animals by executing computer-executable instructions stored on a non-transitory computer-readable medium as claimed in claim 1, wherein the limb rehabilitation device is a limb cast.

4. The method of providing custom limb rehabilitation devices for animals by executing computer-executable instructions stored on a non-transitory computer-readable medium as claimed in claim 1 comprises the steps of:
receiving a diagnosis request through the user interface;
receiving a diagnosis report from a medical practitioner account; and
processing the diagnosis report through the processing device in order to produce the limb rehabilitation device model.

5. The method of providing custom limb rehabilitation devices for animals by executing computer-executable instructions stored on a non-transitory computer-readable medium as claimed in claim 1 comprises the steps of:
receiving a limb designation as the injury data.

6. The method of providing custom limb rehabilitation devices for animals by executing computer-executable instructions stored on a non-transitory computer-readable medium as claimed in claim 1 comprises the steps of:
receiving an image as the injury data.

7. The method of providing custom limb rehabilitation devices for animals by executing computer-executable instructions stored on a non-transitory computer-readable medium as claimed in claim 1 comprises the steps of:
receiving a diagnosis report as the injury data.

8. The method of providing custom limb rehabilitation devices for animals by executing computer-executable instructions stored on a non-transitory computer-readable medium as claimed in claim 1 comprises the steps of:
calculating a plurality of dimensions for the limb rehabilitation device model from the plurality of subject attributes; and
executing the additive manufacturing process according to the plurality of dimensions.

9. The method of providing custom limb rehabilitation devices for animals by executing computer-executable instructions stored on a non-transitory computer-readable medium as claimed in claim 1 comprises the steps of:
receiving a manufacturing progress status from the additive manufacturing unit; and
displaying the manufacturing process status on the user interface.

10. The method of providing custom limb rehabilitation devices for animals by executing computer-executable instructions stored on a non-transitory computer-readable medium as claimed in claim 1 comprises the steps of:
displaying a surgical applications menu on the user interface; and
receiving at least one surgical application selection through the user interface.
